(19) United States
(12) Patent Application Publication
(10) Pub. No.: US 2015/0044195 A1
(43) Pub. Date: Feb. 12, 2015

(54) FVIIa-sTF complexes exhibiting exosite-mediated super activity

(71) Applicant: Novo Nordisk HealthCare AG, Zurich (CH)

(72) Inventors: Henrik Oestergaard, Oelstykke (DK); Anders Laerke Nielsen, Bagsvaerd (DK); Ole Hvilsted Olsen, Broenshoej (DK)

(73) Assignee: NOVO NORDISK HEALTHCARE AG, Zurich (CH)

(21) Appl. No.: 14/385,026

(22) PCT Filed: Mar. 15, 2013
(86) PCT No.: PCT/EP2013/055340

§ 371 Date: Sep. 12, 2014

Related U.S. Application Data
(60) Provisional application No. 61/619,662, filed on Apr. 3, 2012.

(30) Foreign Application Priority Data
Mar. 30, 2012 (EP) 12162483.7

(51) Int. Cl. C12N 9/50

(52) U.S. Cl. CPC C12N 9/50; C12Y 304/21021
USPC 424/94.64; 435/219; 435/352

(57) ABSTRACT

Disclosed are disulphide-linked complexes of a soluble Tissue Factor (sTF) variant of SEQ ID NO:3 comprising the mutation G109C and a Factor VIIa variant of SEQ ID NO. 1, comprising the mutation Q64C and a mutation at position M306 that gives rise to a zymogen-like conformation in the Factor VIIa polypeptide. Said complexes may be used for the treatment of a coagulopathy.

### TECHNICAL FIELD

The current invention relates to pro-coagulant complexes of a Factor VIIa polypeptide and a Tissue Factor polypeptide.

### INCORPORATION BY REFERENCE OF THE SEQUENCE LISTING

The Sequence Listing is 10.878 bytes, was created on 22 Mar. 2012 and is incorporated herein by reference.

### BACKGROUND

In subjects with a coagulopathy, such as in individuals with haemophilia, various steps of the coagulation cascade are rendered dysfunctional due to, for example, the absence or insufficient presence of a coagulation factor. Such dysfunction of one part of the coagulation cascade results in insufficient blood coagulation and potentially life-threatening bleeding or damage to internal organs, such as the joints. Individuals with haemophilia A and B may receive coagulation factor replacement therapy such as exogenous Factor VIII (FVIII) or Factor IX (FIX), respectively. Individuals with haemophilia A and B may develop inhibitors (antibodies) to FVIII or FIX, respectively, in which case treatment with bypassing agents such as exogenous Factor VIIa (FVIIa) may be warranted.

Factor VII (FVII) is a glycoprotein primarily produced in the liver. The mature protein consists of 406 amino acid residues and is composed of four domains as defined by homology. There is an N-terminal Gla domain followed by two epidermal growth factor ((EGF)-like) domains and a C-terminal serine protease domain. FVII circulates in plasma as a single-chain molecule. Upon activation to activated FVII (FVIIa), the molecule is cloven between residues Arg152 and Ile153, resulting in a two-chain protein held together by a disulphide bond. The light chain contains the Gla and EGF-like domains, whereas the heavy chain is the protease domain. FVIIa requires binding to its co-factor, tissue factor (TF), to attain fullbiological activity.

TF is a 263 amino acid integral membrane glycoprotein receptor residing on the cells of the vascular adventitia. It consists of an extracellular part folded into two compact fibronectin type III-like domains (1-219), each stabilized by a single disulphide bond, a transmembrane segment (220-242) and a short cytoplasmic tail (243-263). It serves as the key initiator of coagulation by forming a tight Ca²⁺ dependent complex with FVII, which is captured from circulation upon vascular injury. TF greatly enhances the proteolytic activity of FVIIa towards its physiologic substrates Factor IX and Factor X by serving as a molecular scaffold, by providing the required exosite interactions to its physiological substrates and by inducing conformational changes in the protease domain of FVIIa, resulting in maturation of the active site region of the protease. The activation of FVIIa by TF, which is a result of direct protein-protein interactions, can be mimicked in vitro by saturating FVIIa with a soluble ectodomain of TF, such as sTF(1-219).

EP2007417B1 discloses complexes comprising a FVIIa polypeptide and a soluble TF polypeptide. These complexes have been shown to exhibit a very high proteolytic activity on the phospholipid membrane but this advantageous characteristic is accompanied by a high proteolytic activity in solution and a high amidolytic activity towards small peptide substrates, as well as a fast inhibition by circulating plasma inhibitors, such as antithrombin III (ATM). In an in vivo setting, such complexes maybe inactivated quickly, resulting in a short pharmacokinetic profile.

There is thus a need for complexes comprising a FVIIa polypeptide and a soluble TF polypeptide that exhibit the desirable property of high proteolytic activity on the membrane surface, as well as reduced amidolytic activity and decreased proteolytic activity in solution. Such complexes are, preferably, minimally immunogenic.

### SUMMARY

The invention relates to a disulphide-linked complex of (i) a FVIIa variant of SEQ ID NO: 1 comprising substitution of the amino acid residue Gln64 with Cys and substitution of the amino acid residue Met306 with another naturally occurring amino acid residue and (ii) a soluble Tissue Factor (sTF) variant of SEQ ID NO: 3 comprising substitution of the amino acid residue Gly109 with Cys. The FVIIa variant polypeptide may further comprise a substitution of the amino acid residue Asp309. The invention also relates to a nucleic acid molecule comprising the disulphide-linked complex and a cell that expresses the disulphide complex.

One method of manufacturing the invented disulphide-linked complexes comprises: (i) producing, in a mammalian cell, a Factor VIIa variant of SEQ ID NO: 1 comprising substitution of the amino acid residue Gln64 with Cys and substitution of the amino acid residue Met306 with another naturally occurring amino acid; (ii) producing, in a prokaryotic or eukaryotic cell, asoluble Tissue Factor variant of SEQ ID NO: 3 comprising substitution of the amino acid residue Gly109 with Cys; (iii) labelling the Cys with a heterobifunctional reagent in which one of the functionalities is cysteine reactive; and (iv) cross-linking the soluble Tissue Factor variant to the Factor VIIa variant by means of the second functionality of the heterobifunctional reagent.

A disulphide-linked complex according to the invention may be used as a medicament, particularly for the treatment of a coagulopathy.

### BRIEF DESCRIPTION OF THE DRAWINGS

FIG. 1: Proof of a zymogen-like conformation of the protease domain in the FVIIa(Q64C)(M306D)-sTF(G109C) complex. Carbamoylation of () 150 nMwt-FVIIa (▪) 10 nMwt-FVIIa+100 nMsTF (▴) 152 nMFVIIa(Q64C)(M306D)-sTF(G109C). The species wereincubated with 0.2 M KOCN and residual activity determined at the indicated time-points. The FVIIa(Q64C)(M306D)-sTF(G109C) complex was found to have a carbamoylation profile identical to that of free FVIIa.

FIG. 2: Amidolytic activity towards the S-2288 chromogenic substrate and proteolytic activity towards FX in the absence and presence of 10:90 PS:PC vesicles. The activities are provided as relative numbers with respect to free wt-FVIIa under identical conditions. A 1.8 fold increase in amidolytic activity was found, whilst the proteolytic activity in the absence of vesicles was found to be enhanced 9-fold. In the presence of the phospholipid vesicles, the increase in activity was ˜3000-fold.

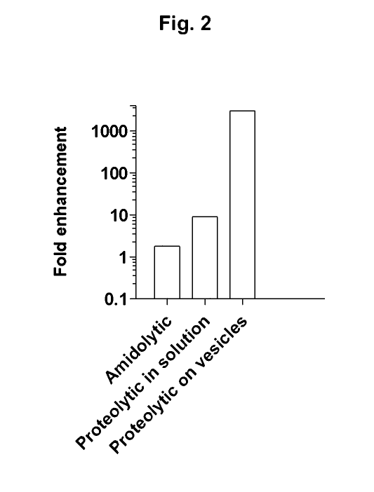

FIG. 3: Results from an in vivo test of the complexes in FVIII knock-out (KO) mice, compared to FVIIa treated FVIII KO mice and wt-mice. Asterisks mark samples that are not statistically different. A modest pro-coagulant effect was seen for the FVIIa Q64C-sTF(1-219) G109C complex (Q64C), while normalisation to wt-levels was seen for both doses of FVIIa Q64C M306D-sTF(1-219) G109C.

### BRIEF DESCRIPTION OF THE SEQUENCES

SEQ ID NO: 1 provides the amino acid sequence (1-406) of native (wild-type) human factor VII. The three-letter indication “GLA” means 4-carboxyglutamic acid (y-carboxyglutamate).

SEQ ID NO: 2 provides the nucleotide sequence of native (wild-type) human factor VII, including the signal peptide (underlined).

SEQ ID NO: 3 provides the amino acid sequence of native (wild-type) human soluble Tissue Factor (1-219).

SEQ ID NO: 4 provides the nucleotide sequence of native (wild-type) human soluble Tissue Factor (1-219) including the signal peptide (underlined).

SEQ ID NOs: 5 to 12 provide the nucleotide sequences of the DNA oligos used for construction of plasmids, as shown in Table 1.

### DESCRIPTION

The present invention relates to disulphide-linked complexes of a Factor VII(a) (FVII(a)) polypeptide and a Tissue Factor (TF) polypeptide. By introducing one or more disulphide-bonds at specific sites in the FVIIa-TF interface, a complex with amidolytic activity comparable to that of wt-FVIIa, when saturated with TF, is obtained.

In the present context, the term “FVII(a)” encompasses the uncloven zymogen, FVII, as well as the cloven and thus activated protease, FVIIa. FVII(a) includes natural allelic variants of FVII(a) that may exist and occur from one individual to another. One wild type human FVII(a)amino acid sequence is provided in SEQ ID NO: 1, as well as in Proc. Natl. Acad. Sci. USA 1986; 83:2412-2416.

The term “FVII(a) polypeptide” herein refers to wild type FVII(a) molecules as well as FVII(a) variants, FVII(a) derivatives and FVII(a) conjugates. Such variants, derivatives and conjugates may exhibit substantially the same, reduced or improved, biological and/or pharmacokinetic activity relative to wild-type human FVIIa.

In the present context, the term “Tissue Factor polypeptide” refers to a polypeptide comprising the soluble ectodomain of Tissue Factor, that is, amino acids 1-219 (in the following referred to as sTF or sTF(1-219)), or a functional variant or truncated form thereof. Preferably, the Tissue Factor polypeptide at least comprises a fragment corresponding to the amino acid sequence 6-209 of Tissue Factor. Particular examples are sTF(6-209), sTF(1-209) and sTF(1-219).

The FVII(a) polypeptide of the above-mentioned complex may be a FVII(a) variant of SEQ ID NO: 1 comprising substitution of the amino acid residue Gln64 with Cys. The TF polypeptide of the complex may be a soluble Tissue Factor (sTF) variant of SEQ ID NO: 3 comprising substitution of the amino acid Gly109 with Cys. The FVII(a) polypeptide further comprises one or more mutations that abolish the allosteric stimulation of FVIIa by TF. A complex with a zymogen-like conformation of the FVIIa protease domain is thus obtained, resulting in a near wild-type amidolytic activity and a low degree of antithrombin III (ATIII) reactivity. For example, the FVIIa polypeptide may further comprise a substitution of the amino acid residue Met306 with another naturally occurring amino acid residue, such as Asp (Biochem. (2001)40, 3251-3256).

The FVIIa polypeptide may comprise a substitution of the amino acid residue Met306 with a naturally occurring polar amino acid residue; that is, Arg, Asn, Asp, Cys, Glu, Gln, His, Lys, Ser, Thr or Tyr.

The FVIIa polypeptide may comprise a substitution of the amino acid residue Met306 with a naturally occurring nonpolar amino acid residue; that is, Ala, Gly, Ile, Leu, Met, Phe, Pro, Trp or Val.

The FVIIa polypeptide may comprise a substitution of the amino acid residue Met306 with a naturally occurring neutral amino acid residue; that is, Ala, Asn, Cys, Gln, Gly, His, Ile, Leu, Met, Phe, Pro, Ser, Thr, Trp, Tyr or Val.

The FVIIa polypeptide may comprise a substitution of the amino acid residue Met306 with a naturally occurring amino acid residue that is acidic at neutral pH; that is, Asp or Glu.

The FVIIa polypeptide may comprise a substitution of the amino acid residue Met306 with a naturally occurring amino acid residue that is basic at neutral pH; that is, Arg, Lys or His.

The FVIIa polypeptide may comprise a substitution of the amino acid residue Met306 with Asp.

The FVIIa polypeptide may comprise a substitution of the amino acid residue Met306 with Ala.

The FVIIa polypeptide may comprise a substitution of the amino acid residue Met306 with Arg.

The FVIIa polypeptide may comprise a substitution of the amino acid residue Met306 with Asn.

The FVIIa polypeptide may comprise a substitution of the amino acid residue Met306 with Cys.

The FVIIa polypeptide may comprise a substitution of the amino acid residue Met306 with Glu.

The FVIIa polypeptide may comprise a substitution of the amino acid residue Met306 with Gln.

The FVIIa polypeptide may comprise a substitution of the amino acid residue Met306 with Gly.

The FVIIa polypeptide may comprise a substitution of the amino acid residue Met306 with His.

The FVIIa polypeptide may comprise a substitution of the amino acid residue Met306 with Ile.

The FVIIa polypeptide may comprise a substitution of the amino acid residue Met306 with Leu.

The FVIIa polypeptide may comprise a substitution of the amino acid residue Met306 with Lys.

The FVIIa polypeptide may comprise a substitution of the amino acid residue Met306 with Met.

The FVIIa polypeptide may comprise a substitution of the amino acid residue Met306 with Phe.

The FVIIa polypeptide may comprise a substitution of the amino acid residue Met306 with Pro.

The FVIIa polypeptide may comprise a substitution of the amino acid residue Met306 with Ser.

The FVIIa polypeptide may comprise a substitution of the amino acid residue Met306 with Thr.

The FVIIa polypeptide may comprise a substitution of the amino acid residue Met306 with Trp.

The FVIIa polypeptide may comprise a substitution of the amino acid residue Met306 with Tyr.

The FVIIa polypeptide may comprise a substitution of the amino acid residue Met306 with Val

The FVIIa polypeptide may comprise a substitution of the amino acid residue Met306 with Ser.

The FVIIa polypeptide may comprise a substitution of the amino acid residue Met306 with Thr.

The FVIIa polypeptide may comprise a substitution of the amino acid residue Met306 with Asn.

In order to destabilise interaction with TF, the FVIIa polypeptide may further comprise substitution of the Asp at position 309 with another naturally occurring amino acid residue, which may be encoded by nucleic acid constructs.

The FVIIa polypeptide may comprise a substitution of the amino acid residue Asp309 with a naturally occurring polar amino acid residue; that is, Arg, Asn, Asp, Cys, Glu, Gln, His, Lys, Ser, Thr or Tyr.

The FVIIa polypeptide may comprise a substitution of the amino acid residue Asp309 with a naturally occurring nonpolar amino acid residue; that is, Ala, Gly, Ile, Leu, Met, Phe, Pro, Trp or Val.

The FVIIa polypeptide may comprise a substitution of the amino acid residue Asp309 with a naturally occurring neutral amino acid residue; that is, Ala, Asn, Cys, Gln, Gly, His, Ile, Leu, Met, Phe, Pro, Ser, Thr, Trp, Tyr or Val.

The FVIIa polypeptide may comprise a substitution of the amino acid residue Asp309 with a naturally occurring amino acid residue that is acidic at neutral pH; that is, Asp or Glu.

The FVIIa polypeptide may comprise a substitution of the amino acid residue Asp309 with a naturally occurring amino acid residue that is basic at neutral pH; that is, Arg, Lys or His.

The FVIIa polypeptide may comprise a substitution of the amino acid residue Asp309 with Asp.

The FVIIa polypeptide may comprise a substitution of the amino acid residue Asp309 with Ala.

The FVIIa polypeptide may comprise a substitution of the amino acid residue Asp309 with Arg.

The FVIIa polypeptide may comprise a substitution of the amino acid residue Asp309 with Asn.

The FVIIa polypeptide may comprise a substitution of the amino acid residue Asp309 with Cys.

The FVIIa polypeptide may comprise a substitution of the amino acid residue Asp309 with Glu.

The FVIIa polypeptide may comprise a substitution of the amino acid residue Asp309 with Gln.

The FVIIa polypeptide may comprise a substitution of the amino acid residue Asp309 with Gly.

The FVIIa polypeptide may comprise a substitution of the amino acid residue Asp309 with His.

The FVIIa polypeptide may comprise a substitution of the amino acid residue Asp309 with Ile.

The FVIIa polypeptide may comprise a substitution of the amino acid residue Asp309 with Leu.

The FVIIa polypeptide may comprise a substitution of the amino acid residue Asp309 with Lys.

The FVIIa polypeptide may comprise a substitution of the amino acid residue Asp309 with Met.

The FVIIa polypeptide may comprise a substitution of the amino acid residue Asp309 with Phe.

The FVIIa polypeptide may comprise a substitution of the amino acid residue Asp309 with Pro.

The FVIIa polypeptide may comprise a substitution of the amino acid residue Asp309 with Ser.

The FVIIa polypeptide may comprise a substitution of the amino acid residue Asp309 with Thr.

The FVIIa polypeptide may comprise a substitution of the amino acid residue Asp309 with Trp.

The FVIIa polypeptide may comprise a substitution of the amino acid residue Asp309 with Tyr.

The FVIIa polypeptide may comprise a substitution of the amino acid residue Asp309 with Val

The FVIIa polypeptide may comprise a substitution of the amino acid residue Asp309 with Ser.

The FVIIa polypeptide may comprise a substitution of the amino acid residue Asp309 with Thr.

The FVIIa polypeptide may comprise a substitution of the amino acid residue Asp309 with Asn.

The FVIIa polypeptide may comprise a substitution of the amino acid residue Met 306 with Asp and a substitution of the amino acid residue Asp309 with Ser.

The FVIIa polypeptide may comprise a substitution of the amino acid residue Met 306 with Ala and a substitution of the amino acid residue Asp309 with Ser.

The FVIIa polypeptide may comprise a substitution of the amino acid residue Met 306 with Ser and a substitution of the amino acid residue Asp309 with Ser.

The FVIIa polypeptide may comprise a substitution of the amino acid residue Met 306 with Thr and a substitution of the amino acid residue Asp309 with Ser.

The FVIIa polypeptide may comprise a substitution of the amino acid residue Met 306 with Asn and a substitution of the amino acid residue Asp309 with Ser.

The FVIIa polypeptide may comprise a substitution of the amino acid residue Met 306 with Asp and substitution of the amino acid residue Asp309 with Ala.

The FVIIa polypeptide may comprise a substitution of the amino acid residue Met 306 with Ala and a substitution of the amino acid residue Asp309 with Ala.

The FVIIa polypeptide may comprise a substitution of the amino acid residue Met 306 with Ser and a substitution of the amino acid residue Asp309 with Ala.

The FVIIa polypeptide may comprise a substitution of the amino acid residue Met 306 with Thr and a substitution of the amino acid residue Asp309 with Ala.

The FVIIa polypeptide may comprise a substitution of the amino acid residue Met 306 with Asn and a substitution of the amino acid residue Asp309 with Ala.

Residues in the FVII(a) protease domain that mayalso be substituted in order to further decrease the amidolytic activity, whilst maintaining a relatively high proteolytic activity, are listed in Table 1, BI and BII of Proc. Nat. Acad. Sci. USA (1996), 93, 14379-14384.

The FVII(a) polypeptide of the above-mentioned complex may be at least 80%, such as at least 85%, such as at least 90%, such as at least 95%, such as at least 96%, such as at least 97%, such as at least 98%, such as at least 99% identical to that represented by SEQ ID NO: 1.

The TF polypeptide of the above-mentioned complex may be at least 80%, such as at least 85%, such as at least 90%, such as at least 95%, such as at least 96%, such as at least 97%, such as at least 98%, such as at least 99% identical to that represented by SEQ ID NO: 3.

The term “identity” as known in the art, refers to a relationship between the sequences of two or more polypeptides, as determined by comparing the sequences. In the art, “identity” also means the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA (1992) 89, 10915-10919 for the BLOSUM 62 comparison matrix) is also used by the algorithm.

Preferred parameters for a peptide sequence comparison include the following: Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

The term “similarity” is a related concept, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% (fraction ( 15/20)). Therefore, in cases where there are conservative substitutions, the degree of similarity between two polypeptides will be higher than the percent identity between those two polypeptides.

The activity of FVII(a)-TF complexes may be tested using a variety of methods that are well-known to the person skilled in the art. Suitable methods include the in vitro solution-based proteolysis assay, the in vitro amidolytic assay, the thromboelastography (TEG) assay, the carbamoylation assay, the inhibition assay and the in vitro antithrombin III inhibition assay that are described in detail in the examples.

As illustrated in the examples, the FVII(a)-TF complexes of the current invention have a reduced amidolytic activity and a decreased proteolytic activity in solution, whilst retaining the desirable property of high proteolytic activity on the membrane surface. Therefore the risk of a recipient developing disseminated intravascular coagulation is minimized. Furthermore, the complexes may have a prolonged circulation time. A further advantage is that the complex is controlled solely by its exosite's specificity which means that cleavage of e.g. the protease-activated receptors (PARs) will be low. A still further advantage of the current complexes is that the mutations that have been introduced are not surface exposed, thus reducing the risk of immunogenicity.

The FVII(a) intermediate of the complexes disclosed herein may be plasma-derived or recombinantly produced, using well known methods of production and purification. The TF intermediate of the complexes disclosed herein may be recombinantly produced using well known methods of production and purification. The degree and location of glycosylation, gamma-carboxylation and other post-translational modifications may vary depending on the chosen host cell and its growth conditions.

The Factor VII polypeptide and the Tissue Factor polypeptide may also be co-expressed in bacteria such as Escherichia coli or in transgenic animals, such as those disclosed in WO 05/075635. The FVII(a) and TF intermediates may then be cross-linked.

In one particularly interesting variant, the method for the preparation of the complex involves the co-expression of the Factor VII polypeptide and the Tissue Factor polypeptide, whereby the covalent link between the two polypeptides can be readily established intracellularly.

One method in which the disulphide complex may be produced comprises (a) transfecting a cell with (i) an expression vector comprising a nucleic acid molecule encoding the Factor VIIa variant of SEQ ID NO:1 as defined herein and expression control regions operatively linked thereto; and (ii) an expression vector comprising a nucleic acid molecule encoding the soluble Tissue Factor variant of SEQ ID NO: 3 as defined herein and expression control regions operatively linked thereto; (b) culturing the transfected cell under conditions for expression of the Factor VII polypeptide and Tissue Factor polypeptide; c) selecting for cells that stably express the complex using the expression control regions of the FVII nucleic acid molecule and d) isolating the expressed complex.

Expression of protein in cells is well-known to the person skilled in the art of protein production. In practicing the method of the invention, the cells are typically eukaryotic cells, more preferably an established eukaryotic cell line, including, without limitation, CHO (e.g., ATCC CCL 61), COS-1 (e.g., ATCC CRL 1650), baby hamster kidney (BHK), and HEK293 (e.g., ATCC CRL 1573; Graham et al., J. Gen. Virol. 36:59-72, 1977) cell lines. A preferred BHK cell line is the tk-ts13 BHK cell line (Waechter and Baserga, Proc. Natl. Acad. Sci. USA 79:1106-1110, 1982), henceforth referred to as BHK 570 cells. The BHK 570 cell line is available from the American Type Culture Collection, 12301 Parklawn Dr., Rockville, Md. 20852, under ATCC accession number CRL 10314. A tk-ts13 BHK cell line is also available from the ATCC under accession number CRL 1632. A preferred CHO cell line is the CHO K1 cell line available from ATCC under accession number CCI61.

Other suitable cell lines include, without limitation, Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1); DUKX cells (CHO cell line) (Urlaub and Chasin, Proc. Natl. Acad. Sci. USA 77:4216-4220, 1980) (DUKX cells also being referred to as DXB11 cells), and DG44 (CHO cell line) (Cell, 33: 405, 1983, and Somatic Cell and Molecular Genetics 12: 555, 1986). Also useful are 3T3 cells, Namalwa cells, myelomas and fusions of myelomas with other cells. In some embodiments, the cells may be mutant or recombinant cells, such as, e.g., cells that express a qualitatively or quantitatively different spectrum of enzymes that catalyze post-translational modification of proteins (e.g., glycosylation enzymes such as glycosyltransferases and/or glycosidases, or processing enzymes such as propeptides) than the cell type from which they were derived. Suitable insect cell lines also include, without limitation, Lepidoptera cell lines, such as Spodoptera frugiperda cells or Trichoplusiani cells (see, e.g., U.S. Pat. No. 5,077,214).

In some embodiments, the cells used in practicing the invention are capable of growing in suspension cultures. As used herein, suspension-competent cells are cells that can grow in suspension without making large, firm aggregates, i.e., cells that are monodisperse or grow in loose aggregates with only a few cells per aggregate. Suspension-competent cells include, without limitation, cells that grow in suspension without adaptation or manipulation (such as, e.g., hematopoietic cells or lymphoid cells) and cells that have been made suspension-competent by gradual adaptation of attachment-dependent cells (such as, e.g., epithelial or fibroblast cells) to suspension growth.

The cells used in practicing the invention may be adhesion cells (also known as anchorage-dependent or attachment-dependent cells). As used herein, adhesion cells are those that need to adhere or anchor themselves to a suitable surface for propagation and growth. In one embodiment of the invention, the cells used are adhesion cells. In these embodiments, both the propagation phases and the production phase include the use of microcarriers. The used adhesion cells should be able to migrate onto the carriers (and into the interior structure of the carriers if a macroporous carrier is used) during the propagation phase(s) and to migrate to new carriers when being transferred to the production bioreactor. If the adhesion cells are not sufficiently able to migrate to new carriers by themselves, they may be liberated from the carriers by contacting the cell-containing microcarriers with proteolytic enzymes or EDTA. The medium used (particularly when free of animal-derived components) should furthermore contain components suitable for supporting adhesion cells; suitable media for cultivation of adhesion cells are available from commercial suppliers, such as, e.g., Sigma.

The cells may also be suspension-adapted or suspension-competent cells. If such cells are used, the propagation of cells may be done in suspension, thus microcarriers are only used in the final propagation phase in the production culture vessel itself and in the production phase. In case of suspension-adapted cells the microcarriers used are typically macroporous carriers wherein the cells are attached by means of physical entrapment inside the internal structure of the carriers. In such embodiments, the eukaryotic cell is typically selected from CHO, BHK, HEK293, myeloma cells, etc.

In one particularly interesting embodiment thereof, the two polypeptides are linked by means of a specific link, more particular by means of a direct link, such as one or more disulphide links between the Factor VII polypeptide and the Tissue Factor polypeptide.

In one embodiment, the method for the preparation of the FVII(a)-TF complex involves production of a cysteine variant of soluble Tissue factor, subsequent labelling of the cysteine in soluble Tissue Factor with a heterobifunctional reagent in which one of the functionalities is cysteine reactive, and finally cross-linking to Factor VIIa by virtue of the second functionality of the reagent. Methods for cloning and expression of cysteine variants of Tissue Factor in E. coli as well as subsequent labelling with a cysteine-specific reagent have been described previously (Stone et al. (1995) Biochem. J., 310, 605-614; Freskgård et al. (1996) Protein Sci., 5, 1521-1540; Owenius et al. (1999) Biophys. J., 77, 2237-2250; Österlund et al. (2001) Biochemistry, 40, 9324-9328). Photo-crosslinking of proteins using heterobifunctional reagents containing one cysteine specific and one photo-activatable functionality have been described by Zhang et al. (1995) Biochem. Biophys. Res. Commun., 217, 1177-1184. Examples of particularly suitable heterobifunctional reagents include p-azidoiodoacetanilide, p-azidophenacyl bromide and p-azidobromoacetanilide,

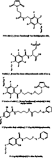

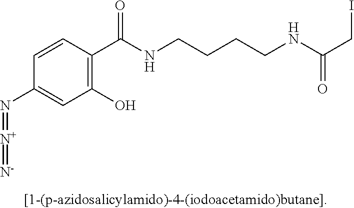 [SMILES: [N-]=[N+]=Nc1ccc(C(=O)NCCCCNC(=O)CI)c(O)c1]

Thus, another method of manufacturing the disulphide complex comprises: (i) producing, in a mammalian cell, the Factor VIIa variant of SEQ ID NO: 1 as defined herein; (ii) producing, in a prokaryotic or eukaryotic cell, asoluble Tissue Factor variant of SEQ ID NO: 3 as defined herein; (iii) labelling the Cys with a heterobifunctional reagent in which one of the functionalities is cysteine reactive; and (iv) cross-linking the soluble Tissue Factor variant to the Factor VIIa variant by means of the second functionality of the heterobifunctional reagent.

FVII(a)-TF complexes of the current invention may be further engineered by adding a half-life extending moiety. The term “half-life extending moiety” is herein understood to refer to one or more chemical groups attached to one or more amino acid site chain functionalities such as —SH, —OH, —COOH, —CONH₂, —NH₂, or one or more N- and/or O-glycan structures and that can increase in vivo circulatory half-life of a number of therapeutic proteins/peptides when conjugated to these proteins/peptides.

Protracting moieties may be added by chemical coupling to endogenous amino acid residues; by coupling to site-specific Cys-mutants; by coupling to introduced non-endogenous amino acids or through modification of the glycans.

A PEG molecule may be attached to any part of the FVII(a) or TF part of the complex, including any amino acid residue or carbohydrate moiety of the FVII(a) or TF polypeptide. This includes but is not limited to PEGylated human Factor VII(a), cysteine-PEGylated human Factor VII(a) and variants thereof. Non-limiting examples of Factor VII derivatives includes glyco PEGylated FVII(a) derivatives as disclosed in WO 03/031464 and WO 04/099231 and WO 02/077218.

In another aspect, the present invention provides compositions and formulations comprising complexes according to the current invention. For example, the invention provides a pharmaceutical composition that comprises one or complexes of the invention, formulated together with a pharmaceutically acceptable carrier.

Accordingly, one object of the invention is to provide a pharmaceutical formulation comprising such a complex which is present in a concentration from 0.25 mg/ml to 250 mg/ml, and wherein said formulation has a pH from 2.0 to 10.0. The formulation may further comprise one or more of a buffer system, a preservative, a tonicity agent, a chelating agent, a stabilizer, or a surfactant, as well as various combinations thereof. The use of preservatives, isotonic agents, chelating agents, stabilizers and surfactants in pharmaceutical compositions is well-known to the skilled person. Reference may be made to Remington: The Science and Practice of Pharmacy, 19th edition, 1995.

In one embodiment, the pharmaceutical formulation is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, to which the physician or the patient adds solvents and/or diluents prior to use.

In a further aspect, the pharmaceutical formulation comprises an aqueous solution of such a complex, and a buffer, wherein the antibody is present in a concentration from 1 mg/ml or above, and wherein said formulation has a pH from about 2.0 to about 10.0.

Based on their reduced proteolytic activity in the absence of a surface, complexes of the current invention may be less prone to auto-proteolysis once formulated, thereby increasing the long-term stability of the formulation.

A complex according to the invention or a pharmaceutical formulation comprising said complex may be used to treat a subject with a coagulopathy.

The term “subject”, as used herein, includes any human or non-human vertebrate individual.

The term “coagulopathy”, as used herein, refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic and are identified by a person skilled in the art.

Non-limiting examples of congenital hypocoagulopathies are haemophiliaA, haemophilia B, Factor VII deficiency, Factor X deficiency, Factor XI deficiency, von Willebrand's disease and thrombocytopenias such as Glanzmann′sthombasthenia and Bernard-Soulier syndrome. Said haemophilia A or B may be severe, moderate or mild. The clinical severity of haemophilia is determined by the concentration of functional units of FIX/FVIII in the blood and is classified as mild, moderate, or severe. Severe haemophilia is defined by a clotting factor level of <0.01 U/ml corresponding to <1% of the normal level, while moderate and mild patients have levels from 1-5% and >5%, respectively. Haemophilia A with “inhibitors” (that is, allo-antibodies against factor VIII) and haemophilia B with “inhibitors” (that is, allo-antibodies against factor IX) are non-limiting examples of coagulopathies that are partly congenital and partly acquired.

A non-limiting example of an acquired coagulopathy is serine protease deficiency caused by vitamin K deficiency; such vitamin K-deficiency may be caused by administration of a vitamin K antagonist, such as warfarin. Acquired coagulopathy may also occur following extensive trauma. In this case otherwise known as the “bloody vicious cycle”, it is characterised by haemodilution (dilution althrombocytopaenia and dilution of clotting factors), hypothermia, consumption of clotting factors and metabolic derangements (acidosis). Fluid therapy and increased fibrinolysis may exacerbate this situation. Said haemorrhage may be from any part of the body.

A non-limiting example of an iatrogenic coagulopathy is an overdosage of anticoagulant medication—such as heparin, aspirin, warfarin and other platelet aggregation inhibitors—that may be prescribed to treat thromboembolic disease. A second, non-limiting example of iatrogenic coagulopathy is that which is induced by excessive and/or inappropriate fluid therapy, such as that which may be induced by a blood transfusion.

In one embodiment of the current invention, haemorrhage is associated with haemophilia A or B. In another embodiment, haemorrhage is associated with haemophilia A or B with acquired inhibitors. In another embodiment, haemorrhage is associated with thrombocytopenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intracranially, intraaurally or intraocularly. In another embodiment, haemorrhage is associated with anticoagulant therapy.

The term “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

Complexes of the invention are typically administered intravenously and may be suitable for prophylactic or therapeutical (on demand) use.

### EMBODIMENTS

The following is a non-limiting list of embodiments of the present invention.

### Embodiment 1

A disulphide-linked complex of (i) a FVIIa variant of SEQ ID NO: 1 comprising substitution of the amino acid residue Gln64 with Cys and substitution of the amino acid residue Met306 with another naturally occurring amino acid residue and (ii) a soluble Tissue Factor (sTF) variant of SEQ ID NO: 3 comprising substitution of the amino acid residue Gly109 with Cys.

### Embodiment 2

The disulphide-linked complex according to embodiment 1, wherein said Met306 is substituted with a naturally occurring polar amino acid residue.

### Embodiment 3

The disulphide-linked complex according to embodiment 1, wherein said Met306 is substituted with a naturally occurring nonpolar amino acid residue.

### Embodiment 4

The disulphide-linked complex according to embodiment 1, wherein said Met306 is substituted with a naturally occurring neutral amino acid residue.

### Embodiment 5

The disulphide-linked complex according to embodiment 1, wherein said Met306 is substituted with a naturally occurring amino acid residue that is acidic at neutral pH.

### Embodiment 6

The disulphide-linked complex according to embodiment 1, wherein said Met306 is substituted with a naturally occurring amino acid residue that is basic at neutral pH.

### Embodiment 7

The disulphide-linked complex according to any one of embodiments 2 or 5, wherein said Met306 is substituted with Asp.

### Embodiment 8

The disulphide-linked complex according to any one of embodiments 3 and 4, wherein said Met306 is substituted with Ala.

### Embodiment 9

The disulphide-linked complex according to any one of embodiments 2 and 4, wherein said Met306 is substituted with Asn.

### Embodiment 10

The disulphide-linked complex according to any one of embodiments 2 and 4, wherein said Met306 is substituted with Ser. Embodiment 11: The disulphide-linked complex according to any one of embodiments 2 or 4, wherein said Met306 is substituted with Thr.

### Embodiment 12

The disulphide-linked complex according to any one of embodiments 1-11, further comprising substitution of the amino acid residue Asp309 with another naturally occurring amino acid residue.

### Embodiment 13

The disulphide-linked complex according to embodiment 12, wherein said Asp309 is substituted with a naturally occurring polar amino acid residue.

### Embodiment 14

The disulphide-linked complex according to embodiment 12, wherein said Asp309 is substituted with a naturally occurring nonpolar amino acid residue

### Embodiment 15

The disulphide-linked complex according to embodiment 12, wherein said Asp309 is substituted with a naturally occurring neutral amino acid residue

### Embodiment 16

The disulphide-linked complex according to embodiment 12, wherein said Asp309 is substituted with a naturally occurring amino acid residue that is acidic at neutral pH.

### Embodiment 17

The disulphide-linked complex according to embodiment 12, wherein said Asp309 is substituted with a naturally occurring amino acid residue that is basic at neutral pH; that is, Arg, Lys or His.

### Embodiment 19

The disulphide-linked complex according to any one of embodiments 14 or 15, wherein said Asp309 is substituted with Ala.

### Embodiment 20

The disulphide-linked complex according to any one of embodiments 13 or 15, wherein said Asp309 is substituted with Ser.

### Embodiment 21

A nucleic acid molecule comprising the disulphide-linked complex according to any one of embodiments 1-20.

### Embodiment 22

A cell that expresses the disulphide-linked complex according to any one of embodiments 1-20.

### Embodiment 23

A method of manufacturing the complex according to any one of embodiments 1-20 comprising: (i) producing, in a mammalian cell, a Factor VIIa variant of SEQ ID NO: 1 comprising substitution of the amino acid residue Gln64 with Cys and substitution of the amino acid residue Met306 with another naturally occurring amino acid; (ii) producing, in a prokaryotic or eukaryotic cell, asoluble Tissue Factor variant of SEQ ID NO: 3 comprising substitution of the amino acid residue Gly109 with Cys; (iii) labelling the Cys with a heterobifunctional reagent in which one of the functionalities is cysteine reactive; (iv) cross-linking the soluble Tissue Factor variant to the Factor VIIa variant by means of the second functionality of the heterobifunctional reagent.

### Embodiment 24

The disulphide-linked complex according to any one of embodiments 1-20 for use as a medicament.

### Embodiment 25

The disulphide-linked complex according to any one of embodiments 1-20 for use in the treatment of a coagulopathy.

### Embodiment 26

The disulphide-linked complex according to any one of embodiments 1-20 for use in the treatment of haemophilia A or B, with or without inhibitors.

### EXAMPLES

The terminology for amino acid substitutions used in the following examples is as follows. The first letter represents the amino acid naturally present at a position of SEQ ID NO:1 or SEQ ID NO:3. The following number represents the position in SEQ ID NO:1 or SEQ ID NO:3. The second letter represents the different amino acid residue that substitutes the naturally occurring amino acid residue. An example is Factor VIIa Q64C, where a glutamine at position 64 of SEQ ID NO:1 is replaced with a cysteine. In another example, sTF(1-219) G109C, the glycine in position 109 of SEQ ID NO: 3 is replaced with a cysteine.

### Materials

D-Phe-Phe-Arg-chloromethyl ketone was purchased from Bachem. Chromogenic Z-D-Arg-Gly-Arg-p-nitroanilide (S-2765), and H-D-Ile-Pro-Arg-p-nitroanilide (S-2288) substrates were obtained from Chromogenix (Sweden). Human plasma-derived factor X (hFX), Factor Xa (hFXa), and factor IXa (hFIXa) were obtained from Enzyme Research Laboratories Ltd. (South Bend, Ind.). Human whole brain Marathon-ready cDNA library was obtained from Clontech (Mountain View, Calif.). p-aminobenzamidine and potassium cyanate were from Sigma-Aldrich. Chromogenic protease substrates S-2288 and S-2765 were from Chromogenix. L-α-phosphatidylcholine (chicken egg) and L-α-phosphatidylserine (porcine brain) from Avanti Polar Lipids were used for the preparation of 10:90 PS:PC vesicles at a concentration of 2.6 mM as described elsewhere (Smith and Morrissey (2004) J. Thromb. Haem., 2, 1155-1162). LMW Heparin sodium salt from porcine intestininal mucosa and Triton X-100 were from Calbiochem. Sheep α-hFVIII (PAHFVIII-S) was from Haematological Technologies. Soluble tissue factor 1-219 (sTF(1-219)) expressed in Escherichia coli was prepared according to published procedures (Freskgård et al. (1996) Protein Sci., 5, 1531-1540). Expression and purification of recombinant factor VIIa was performed as described previously (Thim et al. (1988) Biochemistry, 27, 7785-7793; Persson et al. (1996) FEBS Lett., 385, 241-243). Factor VIIa Q64C-sTF(1-219) G109C was prepared as described below. All other chemicals were of analytical grade or better.

### Example 1

### Construction of DNA Encoding Factor VII Q64C M306D Mutant

The DNA template for the site-directed mutagenesis was pLN174 as disclosed in WO 02/077218. The amino acid of native (wild-type) factor VII is given in SEQ ID NO:1. The DNA sequence of native (wild-type) factor VII including its pre (signal sequence) and pro-regions is given in SEQ ID NO:2.

Plasmid pAeLN023 encoding factor VII Q64C M306D was constructed by QuickChange® Site-Directed Mutagenesis using a mixture of the primers oAeLN023-f, oAeLN023-r, oAeLN024-f and oAeLN024-r, with pLN174 as template according to manufacturer's instructions (Stratagene, La Jolla, Calif.). The correct identity of all cloned sequences was verified by DNA sequencing.

Construction of DNA Encoding sTF(1-219) and sTF(1-219) G109C Mutant

The DNA coding sequence of sTF(1-219) including its signal sequence was amplified from a human whole brain cDNA library (Marathon-ready cDNA; Clontech Laboratories Inc., Mountain View, Calif.) by PCR using Expand High Fidelity PCR system (Roche Diagnostics Corporation, Indianapolis, Ind.) according to manufacturer's recommendations and primers oHOJ1524 and oHOJ152-r, introducing flanking NheI and XhoI restriction sites (primer sequences are listed in Table 1). The purified PCR product was cut with NheI and XhoI and then ligated into the corresponding sites of pCI-neo (Promega, Madison, Wis.) to give pHOJ356.

TABLE 1
DNA oligos used for construction of plasmids.

| Primer | Plasmid | Sequence (5′→3′) |
|---|---|---|
| oAeLN023-f | pAeLN023 | GGGGGCTCCTGCAAGGACTGTCTCCAGTC |
| | | CTATATCTGCTTCTGCCTCCC |
| oAeLN023-r | pAeLN023 | GGGAGGCAGAAGCAGATATAGGACTGGAG |
| | | ACAGTCCTTGCAGGAGCCCCC |
| oAeLN024-f | pAeLN023 | GGTCCTCAACGTGCCCCGTCTAGATACCC |
| | | AGGACTGCCTGCAGC |
| oAeLN024-r | pAeLN023 | GCTGCAGGCAGTCCTGGGTATCTAGACGG |
| | | GGCACGTTGAGGACC |
| oHOJ152-f | pHOJ356 | GGCGGCGGGCTAGCATGGAGACCCCTGCC |
| | | TGGCCCCGG |
| oHOJ152-r | pHOJ356 | CCGCCGCCCTCGAGTTATTCTCTGAATTC |
| | | CCCTTTCTCCTGG |
| oAeLN015-f | pAeLN025 | GGAGACAAACCTCTGCCAGCCAACAATTC |
| | | AGAGTTTTGAACAGGTGGG |
| oAeLN015-r | pAeLN025 | CCCACCTGTTCAAAACTCTGAATTGTTGG |
| | | CTGGCAGAGGTTTGTCTCC |

The amino acid of sTF(1-219) is given in SEQ ID NO:3. The DNA sequence of sTF(1-219) including its signal sequence is given in SEQ ID NO:4.

Plasmid pAeLN025 encoding sTF(1-219) G109C was constructed by QuickChange® Site-Directed Mutagenesis using primers oAeLN015-f and oAeLN015-r and pHOJ356 as template according to manufacturer's instructions (Stratagene, La Jolla, Calif.). The correct identity of all cloned sequences was verified by DNA sequencing.

### Example 2

### Co-Expression of Factor VII Q64C M306D and sTF(1-219) G109C

Factor VIIa Q64C M306D and sTF(1-219) G109C were stably co-expressed in BHK cells as described previously for FVIIa(Thim et al. (1988) Biochemistry, 27, 7785-7793). Briefly, the pAeLN023 and pAeLn025 plasmids were linearized using Acl1 (New England Biolabls) to aid the incorporation into the BHK genome. The linearized plasmids were purified using a PCR plasmid cleanup kit (Sigma). BHK cells were transfected with a 1:1 mixture of the linearized FVII and sTF coding plasmids, using Genejuice (Invitrogen). Stable cell lines were generated by selection with MTX, where resistance was encoded by the FVII encoding plasmid. Stable cell-lines expressing the complexes were grown in DMEM supplemented with 10% FCS, 1% penicillin/streptomycin and vitamin K₁ to 5 ppm (Sigma) required for post-translational gamma-carboxylation of factor VII. The selection was continued until all cells in a transfection-control were dead. The cells were seeded in 500 ml 10-layer culture flasks and grown until they were confluent. The cells were harvested with 4-5 day intervals for a total of five harvests. Cells were removed by centrifugation at 250 g and the harvests were stored at −80° C. until purification. The resulting stable polyclonal cell-lines all had growth-rates comparable to the wild-type strains.

### Example 3

### Purification of Factor VII Q64C M3060-sTF(1-219) G109C Complex

Conditioned medium to which CaCl₂ had been added to a concentration of 10 mM was loaded onto a 40-ml column containing the monoclonal antibody F1A2 (Novo Nordisk A/S, Bagsvaerd, Denmark) coupled to CNBr-activated Sepharose 4B (Amersham Biosciences, GE Healthcare). The column was equilibrated with 50 mM HEPES, 100 mM NaCl, 10 mM CaCl₂, pH 7.5. After washing with equilibration buffer and equilibration buffer containing 2 M NaCl, bound material was eluted with equilibration buffer containing 10 mM EDTA instead of CaCl₂. Calcium chloride was subsequently added to the collected peak fraction to a final concentration of 20 mM.

To remove small amounts of free factor VIIa Q64C M306D, the preparation was passed over a 1-ml HiTrap NHS column (GE Healthcare) to which 4 mg sTF(1-219) had been coupled according to manufacturer's instructions. Prior to loading, the column was equilibrated in 50 mM HEPES, 100 mM NaCl, 10 mM CaCl₂, pH 7.5. The flow through containing factor VII F40C-sTF(1-219) V207C complex, and devoid of detectable free factor VII F40C and sTF(1-219) V207C, was collected.

To promote activation of the factor VII Q64C M306D-sTF(1-219) G109C complex, human factor IXa was added to a final concentration of 0.04 mg/ml. After complete activation as verified by reducing SDS-PAGE, factor VIIa Q64C M306D-sTF(1-219) G109C complex was purified by F1A2 Sepharose 4B affinity chromatography as described above, except that a 20-ml column was used and the equilibration buffer was 10 mM MES, 100 mM NaCl, 10 mM CaCl₂, pH 6.0. The final protein preparation was stored in aliquots at −80° C.

### SDS-PAGE Analysis

Factor VIIa Q64C M306D-sTF(1-219) G109C complex (approx 3 μg) was analyzed by non-reducing and reducing SDS-PAGE on a 4-12% Bis-TrisNuPAGE® gel (Invitrogen Life Technologies, Carlsbad, Calif.) run at 200 V for 35 min in MES buffer (Invitrogen Life Technologies, Carlsbad, Calif.) according to manufacturer's recommendations. Gels were washed with water and stained with Simply Blue™ SafeStain (Invitrogen Life Technologies, Carlsbad, Calif.) according to manufacturer's recommendations.

The complex was obtained in good purity and based on the reducing SDS_page the activation of the complex was found to be complete and both sTF and FVII was found to be fully glycosylated.

### Example 4

### Active-Site Titration Assay

Active site concentrations of factor VIIa Q64C M306D-sTF(1-219) G109C was determined from the irreversible loss of amidolytic activity upon titration with sub-stoichiometric levels of D-Phe-Phe-Arg-chloromethyl ketone (FFR-cmk) essentially as described elsewhere (Bock P. E. (1992) J. Biol. Chem., 267, 14963-14973). Briefly, each protein was diluted into 50 mM HEPES, 100 mMNaCl, 10 mM CaCl₂, 0.01% Tween 80, pH 7.0 buffer to an approximate concentration of 400 nM. Diluted protein (50 μl) was then combined with 50 μl 0-5 μM FFR-cmk (freshly prepared in buffer from a FFR-cmk stock dissolved in DMSO and stored at −80° C.). After overnight incubation at room temperature, residual amidolytic activity was measured. The activity assay was carried out in polystyrene microtiter plates (Nunc, Denmark) in a final volume of 200 μl assay buffer (50 mM HEPES, 100 mMNaCl, 5 mM CaCl₂, 0.01% Tween 80, pH 7.4) containing approx. 100 nM factor VIIa Q64C G109C-sTF(1-219) G109C complex, corresponding to four-fold dilutions of the samples. After 15 min pre-incubation at room temperature, 1 mM chromogenic substrate S-2288 was added and the absorbance monitored continuously at 405 nm for 10 min in a SpectraMax™ 340 microplate spectrophotometer equipped with SOFTmax PRO software (v2.2; Molecular Devices Corp., Sunnyvale, Calif.). Amidolytic activity was reported as the slope of the linear progress curves after blank subtraction. Active site concentrations were determined by extrapolation to zero activity, corresponding to the minimal concentration of FFR-cmk completely abolishing amidolytic activity.

The active-site titration was found to correspond to within 10% of the concentration as determined by A280 absorbance.

### Example 5

### In Vitro Amidolytic Assay

Native (wild-type) factor VIIa, with and without sTF(1-219), FVIIa Q64C-sTF(1-219) G109C and factor VIIa Q64C M306D-sTF(1-219) G109C were assayed in parallel to directly compare their specific activities. The assay was carried out in a microtiter plate (Nunc, Denmark). Factor VIIa (150 nM), Factor VIIa (10 nM) and sTF(1-219) (100 nM), Factor VIIa Q64C-sTF(1-219) G109C (10 nM) and Factor VIIa Q64C M306D-sTF(1-219) G109C (150 nM) in a total volume of 180 μl in 50 mM HEPES, 100 mM NaCl, 5 mM CaCl₂, 0.01% Tween 80, pH 7.4 buffer. The activity was determined by addition of 1 mM H-D-Ile-Pro-Arg-p-nitroanilide (S-2288). The absorbance at 405 nm was measured continuously in a SpectraMax™ 340 microplate spectrophotometer equipped with SOFTmax PRO software (v2.2; Molecular Devices Corp., Sunnyvale, Calif.). Specific amidolytic activities were determined as the slope of the linear progress curves after blank subtraction divided by the protein concentration in the assay in the case of Factor VIIa, for the other samples the data were fitted to a Michaelis-Menten model and the k_{cat}/K_{M} was explicitly calculated. From this, the ratio between the specific proteolytic activities of factor VIIa-sTF complexes and wild-type factor VIIa were derived as shown in Table 2.

Consistent with the introduction of the M306D mutation, the FVIIa Q64C M306D-sTF(1-219) G109C complex was found to have an amidolytic activity only 1.7-fold higher than that of wt-FVIIa and 25-fold lower than that of the FVIIa Q64C-sTF(1-219) G109C complex. This suggested that the protease domain of FVIIa in the FVIIa Q64C M306D-sTF(1-219) G109C complex is maintained in a zymogen-like conformation.

TABLE 2
Relative amidolytic activities in the
described in vitroamidolytic assay

| | Relative amidolytic |
|---|---|
| Protein | activity (ratio) |
| wt-Factor VIIa | 1 |
| wt-FVIIa and sTF(1-219) | 49 |
| FVIIa Q64C-sTF(1-219) G109C | 51 |
| FVIIa Q64C M306D-sTF(1-219) G109C | 1.7 |

### Example 6

### Carbamoylation Assay

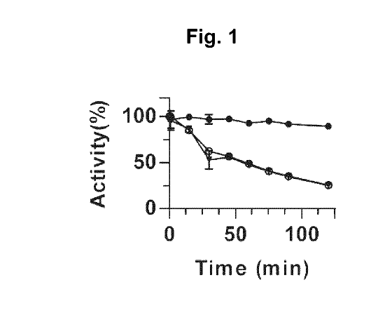

Native (wild-type) factor VIIa, with and without sTF(1-219) and factor VIIa Q64C M306D-sTF(1-219) G109C were assayed in parallel to directly compare the burial of their N-termini from their reactivity with potassium cyanate (Stark et. al Biochemistry 4, 1030-1036 (1965)). The assay was carried out in a microtiter plate (Nunc, Denmark) by incubation of Factor VIIa (1.5 μM), Factor VIIa and sTF(1-219) (100 nM+1 μM), and Factor VIIa Q64C M306D-sTF G109C (1.52 μM) with 0.2 M KOCN at ambient temperature. 20 μl samples were drawn from the reactions at 15 min intervals and diluted 10-fold in assay buffer containing 1 mM S-2288. The absorbance at 405 nm was measured continuously in a SpectraMax™ 340 microplate spectrophotometer equipped with SOFTmax PRO software (v2.2; Molecular Devices Corp., Sunnyvale, Calif.). Initial velocities, reported as the slope of the linear progress curves after blank subtraction divided by the protein concentration in the assay, were plotted as a function of time, see FIG. 1.

The assay revealed that the rate of carbamoylation of the FVIIa Q64C M306D-sTF(1-219) G109C complex was virtually identical to that of FVIIa. This indicated that the degree of insertion of the N-terminus into the activation pocket in the protease domain of the complex was identical to that of free wt-FVIIa. Accordingly, the protease domain of FVIIa Q64C M306D-sTF(1-21) G109C predominantly exists in a zymogen-like conformation, similar to free wt-FVIIa.

### Example 7

### In Vitro Solution-Based Proteolysis Assay

Native (wild-type) factor VIIa, with and without sTF(1-219), FVIIa Q64C-sTF(1-219) G109C and factor VIIa Q64C M306D-sTF(1-219) G109C were assayed in parallel to directly compare their specific activities. The assay was carried out in a microtiter plate (Nunc, Denmark). Factor VIIa (600 nM), Factor VIIa (10 nM) and sTF(1-219) (100 nM), Factor VIIa Q64C-sTF(1-219) G109C (10 nM) and Factor VIIa Q64C M306D-sTF(1-219) G109C (150 nM) were incubated with varying human Factor X concentrations (0-0.2 μM) in 100 μl 50 mM HEPES, 100 mM NaCl, 5 mM CaCl₂, 0.01% Tween 80, pH 7.4. The mixtures were incubated for 20 min at ambient temperature. Factor X activation was subsequently stopped by the addition of 50 μl 50 mM HEPES, 100 mMNaCl, 40 mM EDTA, 0.01% Tween 80, pH 7.4. The amount of FXa generated was measured by addition of 50 μl of the chromogenic substrate Z-D-Arg-Gly-Arg-p-nitroanilide (S-2765) to a final concentration 0.5 mM. The absorbance at 405 nm was measured continuously in a SpectraMax™ 340 microplate spectrophotometer equipped with SOFTmax PRO software (v2.2; Molecular Devices Corp., Sunnyvale, Calif.). Specific proteolytic activities, reported as the slope of the linear progress curves after blank subtraction divided by the protein concentration in the assay, and were used to calculate the ratio between the specific proteolytic activities of factor VIIa-sTF complex and wild-type factor VIIa as shown in Table 3.

As predicted, judging by the zymogen-like features of the FVIIa Q64C M306D-sTF(1-219) G109C complex, the proteolytic activity of the complex in solution was only 9-fold higher than wt-FVIIa and about 30-fold reduced compared to FVIIa Q64C-sTF(1-219) G109C.

TABLE 3
Relative proteolytic activities as described in
the solution based in vitro proteolysis assay

| | Relative proteolytic |
|---|---|
| Protein | activity (ratio) |
| wt-Factor VIIa | 1 |
| wt-FVIIa and sTF(1-219) | 277 |
| FVIIa Q64C-sTF(1-219) G109C | 271 |
| FVIIa Q64C M306D-sTF(1-219) G109C | 9 |

### Example 8

### In Vitro Proteolysis Assay with Phospholipids

Native (wild-type) factor VIIa, with and without sTF(1-219), FVIIa Q64C-sTF(1-219) G109C and factor VIIa Q64C M306D-sTF(1-219) G109C were assayed in parallel to directly compare their specific activities. The assay was carried out in a microtiter plate (Nunc, Denmark). Factor VIIa (150 nM), Factor VIIa (5 pM) and sTF(1-219) (100 nM), Factor VIIa Q64C-sTF(1-219) G109C (5 pM) and Factor VIIa Q64C M306D-sTF(1-219) G109C (30 pM) were incubated with varying human Factor X concentrations (0-500 nM) in 100 μl 50 mM HEPES, 100 mM NaCl, 5 mM CaCl₂, 1 mg/ml BSA, pH 7.4, containing 250 μM 10:90 phospholipid vesicles. The mixtures were incubated for 10 min at ambient temperature. Factor X activation was subsequently stopped by the addition of 50 μl 50 mM HEPES, 100 mMNaCl, 40 mM EDTA, 0.01% Tween 80, pH 7.4. The amount of FXa generated was measured by addition of 50 μl of the chromogenic substrate Z-D-Arg-Gly-Arg-p-nitroanilide (S-2765) to a final concentration 0.5 mM. The absorbance at 405 nm was measured continuously in a SpectraMax™ 340 microplate spectrophotometer equipped with SOFTmax PRO software (v2.2; Molecular Devices Corp., Sunnyvale, Calif.). Specific proteolytic activities were determined as the slope of the linear progress curves after blank subtraction divided by the protein concentration in the assay in the case of Factor VIIa, for the other samples the data were fitted to a Michaelis-Menten model and the k_{cat}/K_{M} was explicitly calculated. From this, the ratio between the specific proteolytic activities of factor VIIa-sTF complexes and wild-type factor VIIa were derived as shown in Table 4.

These results show that despite the amidolytic and proteolytic activities of FVIIa Q64C M306D-sTF(1-219) G109C in solution being comparable to FVIIa, the complex was significantly (about 2400-fold) more active than FVIIa in the presence of a phospholipid surface. Thus, it appears that macromolecular substrate interactions involving regions outside the active site are able to largely compensate for the zymogen-like features of the complex when located on a phospholipid membrane but not in solution. Altogether, these data demonstrate that the proteolytic activity of FVIIa Q64C M306D—sTF(1-219) G109C complex exhibits a significant membrane dependency.

TABLE 4
Relative proteolytic activities in the in
vitro proteolysis assay with PS:PC vesicles

| | Relative proteolytic |
|---|---|
| Protein | activity (ratio) |
| wt-Factor VIIa | 1 |
| wt-FVIIa and sTF(1-219) | 87000 |
| FVIIa Q64C-sTF(1-219) G109C | 78000 |
| FVIIa Q64C M306D-sTF(1-219) G109C | 2400 |

### Example 9

### In Vitro Antithrombin III Inhibition Assay

The inhibition of the complexes by Antithrombin III (ATIII) was determined under pseudo-first order conditions as described elsewhere (Olson et al. (1993), Methods Enzymol. 222, 525-559). Briefly, the assay was conducted in 100 μl volume in 20 mMHepes, 100 mMNaCl, 10 mM CaCl2, 0.01% Tween-80 pH 7.4 by mixing Factor VIIa (200 nM), Factor VIIa and sTF (20 nM+200 nM), Factor VIIa Q64C-sTF(1-219) G109C (20 nM) and Factor VIIa Q64C M306D-sTF(1-219) G109C (200 nM) with low molecular weight heparin (25 μM) followed by pre-incubation for 10 min at ambient temperature. ATIII (2.5 μM) was added at varying intervals to separate rows in the 96 well plate. The assay was quenched after the last addition by the addition of 80 μl 1 mg/ml polybrene, followed by the addition of 20 μl S-2288 (1 mM) and the absorbance monitored continuously at 405 nm for 10 min in a SpectraMax™ 340 microplate spectrophotometer equipped with SOFTmax PRO software (v2.2; Molecular Devices Corp., Sunnyvale, Calif.). Amidolytic activity was determined as the slope of the linear progress curves after blank subtraction. The data were fitted to a first-order exponential decay, divided by the ATIII concentration and the resulting pseudo-first order rate constants are shown in Table 5.

Consistent with the results shown in Table 2, the FVIIa Q64C M306D-sTF(1-219) G109C complex was found to exhibit a significantly reduced rate of inhibition, compared to FVIIa Q64C-sTF(1-219) G109C. Since inhibition by antithrombin constitutes a major clearance pathway of FVIIa in vivo, these data suggest that the half-life of FVIIa Q64C M306D-sTF(1-219) G109C complex in circulation will be longer than that of FVIIa Q64C-sTF(1-219) G109C

TABLE 5
Pseudo first-order rate constants of ATIII inihbition

| | Relative first-order |
|---|---|
| Protein | rate constants(ratio) |
| wt-Factor VIIa | 1 |
| wt-FVIIa and sTF(1-219) | 44 |
| FVIIa Q64C-sTF(1-219) G109C | 44 |
| FVIIa Q64C M306D-sTF(1-219) G109C | 1.6 |

### Example 10

### In Vitro Whole-Blood Based Coagulation Assay

The effect of the Factor VIIa Q64C M306D-sTF G109C complex relative to wt-Factor VIIa and FVIIa Q64C-sTF G109C in Factor VIII deficient whole blood was investigated. Briefly, the assay was conducted using freshly drawn blood from healthy volunteers stabilized by addition of sodium citrate (3.2%). The blood was made FVIII deficient by addition of 0.1 mg/ml sheep anti-FVIII antibody (Haematological Technologies). Samples (15 μl+15 μl buffer) was added to the blood (480 μl), the mixture was gently mixed by turning over the tube. Of this mixture 340 μl was transferred to the cup of a Thrombelastograph TEG® 5000 Hemostasis Analyzer, to which 20 μl 15.5 mM CaCl₂ had been added. The assay was run for 3 h at ambient temperature after which it was terminated discontinously. The clot-times were extracted and the apparent EC₅₀ values are listed in Table 6.

As found in the membrane-dependent proteolytic assay, the FVIIa Q64C M306D-sTF(1-219) G109C complex exhibited significantly increased activity (as measured by the EC₅₀ value) compared to wt FVIIa. This indicates that the molecule may be useful in bypass-treatment of haemophilias A and B.

TABLE 6
Apparent EC₅₀ values from the whole-blood based assay

| | | Number of |
|---|---|---|
| Protein | App. EC₅₀ (pM) | donors |
| wt-Factor VIIa | 396 | 3 |
| FVIIa Q64C-sTF(1-219) G109C | 0.10 | 3 |
| FVIIa Q64C M306D-sTF(1-219) G109C | 4.4 | 3 |

### Example 11

### Thromboelastography in Murine FVIII KO Blood

Before initiating in vivo experiments, the effect of FVIIa and FVIIa Q64C M306-sTF(1-219) G109C in murine blood was assayed using thromboelastography. The effect on the whole blood clotting profile was obtained by thromboelastography and the parameters describing the initiation (clotting time) and propagation phase (angle) of the clot formation were analysed. Citrate stabilized blood was collected from the retro-orbital venous plexus. The first few drops of blood were discharged and only free floating blood was collected. All blood samples were collected under isoflurane anaesthesia. In vitro concentration response curves for rFVIIa analogues was obtained by adding 7 μL of test compound (buffer comp) to 105 μL citrated stabilised blood in pre-warmed curvets. Coagulation was initiated by re-calcification of the samples (7 μL CaCl₂, final Ca²⁺ concentration 11 mM). The thromboelastographic response was measured until the first of maximal thrombus formation or one hour, by ROTEM® delta (ROTEM, Munich, Germany) using the minicuvetes.

The FVIIa Q64C M306D-sTF(1-219) G109C complex was found to have significantly increased activity in this assay compared to FVIIa. The numbers obtained were used to select appropriate dosing ranges for an in vivo study.

TABLE 7
EC₅₀ values from the whole-blood based assay in murine blood

| Protein | EC₅₀ (nM) |
|---|---|
| wt-Factor VIIa | 5.4 |
| FVIIa Q64C-sTF(1-219) G109C | 0.0059 |
| FVIIa Q64C M306D-sTF(1-219) G109C | 0.032 |

### Example 12

### In Vivo Effect

To evaluate the potential of FVIIa Q64C M306D-sTF(1-219) G109C as a compound for treating haemophilia, the compound was tested in FVIII knock-out mice as described in the following. Haemophilia mice (Factor VIII knockout mice) were originally obtained from (Bi et al (1995) Nat Genet 10, 119-121) and bred at Taconic (Ry, Denmark). C57Bl/6J mice were obtained from Taconic. The animals were between 12 and 16 weeks old, with an equal distribution of males and females. The effect of wt-FVIIa and the FVIIa analogues in the tail bleeding model was investigated. In brief, mice were anaesthetised with isoflurane (1.5%; 0.5 L/hr O₂ and 0.7 L/hr N₂O) and the tail was amputated 4 mm proximal to the tip five minutes after administration of wt-FVIIa, FVIIa Q64C-sTF G109C, or FVIIa Q64C M306D-sTF(1-219) G109C (buffer comp). The tail was placed in 37° C. saline and the blood loss collected over a 30 minute period. All test substances were administered intravenously (10 mL/kg). The effect on the blood was compared by a one-way ANOVA, followed by Bonferroni test for multiple comparisons to compare the effect of treatment with that of the vehicle control and the results in wild type mice.

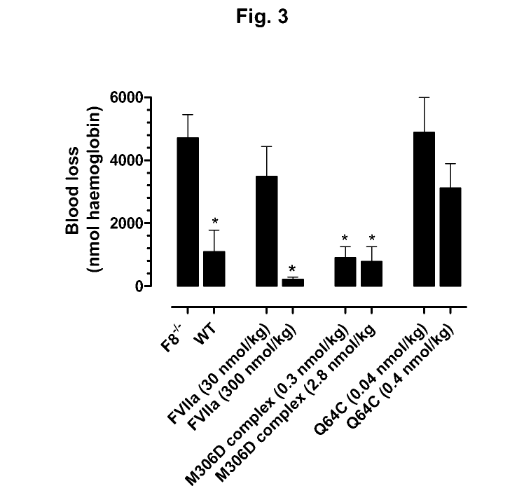

The resulting data are shown in FIG. 3. It was found that the FVIIa Q64C-sTF G109C complex only exhibited a minor effect in murine blood when dosed at a concentration which corresponded to 15 mg/ml FVIIa (300 nmol/kg). This finding could be due to rapid clearance after administration by antithrombin III.

In contrast, the FVIIa Q64C M306D-sTF(1-219) G109C complex was found to normalize the blood-loss to that of a wild-type mice, when administered at a dose 1000-fold lower than the corresponding wt-FVIIa dose. These data provide in vivo proof of concept of the beneficial effect of rendering the FVIIa protease domain in a zymogen-like conformation as the membrane dependent action of the FVIIa Q64C M306D-sTF(1-219) G109C complex allows it to exert its action at the site of injury whilst preventing rapid clearance of the complex by endogenous inhibitors.

Whilst certain features of the invention are illustrated and described herein, many modifications, substitutions, changes, and equivalents now occur to those of ordinary skill in the art. It is, therefore, to be understood that the appended claims are intended to cover all such modifications and changes as fall within the true spirit of the invention.

1. A disulphide-linked complex of (i) a FVIIa variant of SEQ ID NO: 1 comprising substitution of the amino acid residue Gln64 with Cys and substitution of the amino acid residue Met306 with another naturally occurring amino acid residue and (ii) a soluble Tissue Factor (sTF) variant of SEQ ID NO: 3 comprising substitution of the amino acid residue Gly109 with Cys.

2. The disulphide-linked complex according to claim 1, wherein said Met306 is substituted with a naturally occurring polar amino acid residue.

3. The disulphide-linked complex according to claim 2, wherein said Met306 is substituted with Asp.

4. The disulphide-linked complex according to claim 1, wherein said Met306 is substituted with a naturally occurring nonpolar amino acid residue.

5. The disulphide-linked complex according to claim 4, wherein said Met306 is substituted with Ala.

6. The disulphide-linked complex according to claim 1, wherein said Met306 is substituted with a naturally occurring neutral amino acid residue.

7. The disulphide-linked complex according to claim 6, wherein said Met306 is substituted with Asn, Ser or Thr.

8. The disulphide-linked complex according to claim 1, wherein said Met306 is substituted with a naturally occurring amino acid residue that is acidic at neutral pH.

9. The disulphide-linked complex according to claim 1, wherein said Met306 is substituted with a naturally occurring amino acid residue that is basic at neutral pH.

10. The disulphide-linked complex according to claim 1, further comprising substitution of the amino acid residue Asp309 with another naturally occurring amino acid residue.

11. The disulphide-linked complex according to claim 10, wherein said Asp309 is substituted with Ala or Ser.

12. A cell that expresses the disulphide-linked complex according to claim 1.

13. A method of manufacturing the complex according to claim 1 comprising:
(i) producing, in a mammalian cell, a Factor VIIa variant of SEQ ID NO: 1 comprising substitution of the amino acid residue Gln64 with Cys and substitution of the amino acid residue Met306 with another naturally occurring amino acid;
(ii) producing, in a prokaryotic or eukaryotic cell, a soluble Tissue Factor variant of SEQ ID NO: 3 comprising substitution of the amino acid residue Gly109 with Cys;
(iii) labelling the Cys with a heterobifunctional reagent in which one of the functionalities is cysteine reactive;
(iv) cross-linking the soluble Tissue Factor variant to the Factor VIIa variant by means of the second functionality of the heterobifunctional reagent.

14. (canceled)

15. (canceled)

16. A method of treating a coagulopathy in a subject, comprising administering the disulphide-linked complex of claim 1 to said subject.


SEQUENCE LISTING (12 sequences)

SEQ ID NO: 1 - protein, 406 aa, Homo sapiens
  MOD_RES (6)..(7): Xaa = GLA
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - DNA, 1335 bp, Homo sapiens
atggtctcccaggccctcaggctcctctgccttctgcttgggcttcagggctgcctggctgcagtcttcgtaacccaggaggaagcccaaggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctag

SEQ ID NO: 3 - protein, 219 aa, Artificial Sequence
  Synthetic
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 4 - DNA, 756 bp, Artificial Sequence
  Synthetic
atggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaataa

SEQ ID NO: 5 - DNA, 50 bp, Artificial Sequence
  Synthetic
gggggctcctgcaaggactgtctccagtcctatatctgcttctgcctccc

SEQ ID NO: 6 - DNA, 50 bp, Artificial Sequence
  Synthetic
gggaggcagaagcagatataggactggagacagtccttgcaggagccccc

SEQ ID NO: 7 - DNA, 44 bp, Artificial Sequence
  Synthetic
ggtcctcaacgtgccccgtctagatacccaggactgcctgcagc

SEQ ID NO: 8 - DNA, 44 bp, Artificial Sequence
  Synthetic
gctgcaggcagtcctgggtatctagacggggcacgttgaggacc

SEQ ID NO: 9 - DNA, 38 bp, Artificial Sequence
  Synthetic
ggcggcgggctagcatggagacccctgcctggccccgg

SEQ ID NO: 10 - DNA, 42 bp, Artificial Sequence
  Synthetic
ccgccgccctcgagttattctctgaattcccctttctcctgg

SEQ ID NO: 11 - DNA, 48 bp, Artificial Sequence
  Synthetic
ggagacaaacctctgccagccaacaattcagagttttgaacaggtggg

SEQ ID NO: 12 - DNA, 48 bp, Artificial Sequence
  Synthetic
cccacctgttcaaaactctgaattgttggctggcagaggtttgtctcc